(19) United States
(12) Patent Application Publication
(10) Pub. No.: US 2024/0058216 A1
(43) Pub. Date: Feb. 22, 2024

(54) DISPENSER CONFIGURED FOR ASEPTIC TRANSFER OF A LIQUID PRODUCT

(71) Applicant: Oyster Point Pharma, Inc., Princeton, NJ (US)

(72) Inventors: John K. LOKHNAUTH, Fair Lawn, NJ (US); Eric C. EHRNSPERGER, New City, NY (US); Travis D. BUEL, Hickman, NE (US); Jason M. LEPREE, Demarest, NJ (US); George J. DONATO, Swarthmore, PA (US); Jeffrey Alan NAU, Pennington, NJ (US); Peter A. SMITH, Cary, NC (US); Joseph Vincent RANALLETTA, Greenville, SC (US); Eli B. NICHOLS, Jacksonville, FL (US)

(21) Appl. No.: 17/889,285

(22) Filed: Aug. 16, 2022

(51) Int. Cl. A61J 1/14

(52) U.S. Cl. CPC A61J 1/14; A61J 1/2096

(57) ABSTRACT

A drug delivery system includes a container comprising a luer compartment and a fluid compartment, a luer fitting arranged within the luer compartment and in fluidic communication with the fluid compartment, and a dispenser in fluidic communication with the fluid compartment. A method of aseptically transferring a drug to such a drug delivery system may include providing a vial comprising the drug to be transferred, withdrawing a volume of the drug from the vial with a syringe, coupling the syringe to a drug delivery system via the luer fitting, transferring the drug from the syringe to the drug delivery system, and decoupling the syringe from the drug delivery system.

### TECHNICAL FIELD

This invention relates generally to the field of storage and/or delivery of fluids (e.g., drugs).

### BACKGROUND

In many instances, drugs or other medications are transported and/or stored in a form that is different than its form when administered to a patient and must be prepared in in a certain manner before it can be administered to the patient. For example, a lyophilized drug may be reconstituted into a liquid, or a frozen drug may be thawed. Such preparation of the drug typically occurs in a standalone vial, and then the prepared drug may be accessed with a syringe or other suitable device.

In some instances, the prepared drug must be administered to a patient in a particular manner using a designated type of drug delivery device (e.g., eyedropper, nasal spray pump). In these instances, the drug is often prepared in a separate vial and must be subsequently transferred to the drug delivery device for administration. However, this transfer process is risky and may pose challenges to a user. For example, if the transfer process is not handled properly, it may lead to contamination and/or other degradation of the prepared drug. Accordingly, special training may thus be required to properly transfer the drug from a vial to a drug delivery device. Untrained patients or other consumers may find it difficult or confusing to properly handle the drug, which may lead to contamination, a reduction in drug efficacy, danger to the patient, and/or drug product wastage.

Thus, there is a need for a new and improved system and method for aseptic transfer of a liquid product for drug delivery and/or the like.

### SUMMARY

Generally, in some variations, a drug delivery system may include a container including a luer compartment and a fluid compartment, a luer fitting arranged within the luer compartment and in fluidic communication with the fluid compartment, and a dispenser in fluidic communication with the fluid compartment. The luer fitting may, for example, include a luer valve configured to receive fluid from a syringe or other fluid dispensing device. The luer fitting may be coupled to the luer compartment via a fluidic seal. In some variations, the system may further include a cap configured to enclose the luer fitting within the luer compartment. For example, the cap may be configured to couple to the container.

The luer compartment and the fluid compartment may be arranged in any suitable portions or locations of the container. For example, in some variations the luer compartment and the fluid compartment may be arranged on opposite ends of the container (e.g., the luer compartment near a base portion of the container, and the fluid compartment near a neck portion of the container). In some variations, the container may include a conduit extending between the luer compartment and the fluid compartment, such as for transporting fluid from the luer compartment to the fluid compartment. For example, at least a portion of the luer fitting may be arranged within the conduit to transport fluid from the luer compartment to the fluid compartment.

In some variations, the dispenser may be coupled to the container. For example, the dispenser may be directly coupled to the container, such as via a snap fit or threaded interface between the container and the dispenser. Alternatively, the dispenser may be indirectly coupled to the container through an adapter configured to intervene between the container and the dispenser, where the adapter includes a first interface configured to couple to the container and a second interface configured to couple to the dispenser. In some variations, the first interface and the second interface may be different. For example, one of the first and second interfaces may include a snap fit interface, while the other of the first and second interfaces may include a threaded interface.

The drug delivery system may include any suitable kind of dispenser. For example, in some variations the dispenser may include an eyedropper. As another example, the dispenser may include a nasal spray pump. Other aspects of the container may be configured depending on the type of dispenser. For example, in some variations, the container may include a flexible material (e.g., in variations in which the dispenser includes an eyedropper, such that the container may be squeezed to urge drops of fluid out of the eyedropper).

Generally, a method of aseptically transferring a drug to a drug delivery system may include preparing and/or otherwise providing a vial comprising the drug to be transferred, withdrawing a volume of the drug from the vial with a syringe, coupling the syringe to a drug delivery system via a luer fitting, transferring the drug from the syringe to the drug delivery system, and decoupling the syringe from the drug delivery system. In some variations, the method may further include covering the luer fitting of the drug delivery system with a cap, such as after the syringe has been decoupled from the drug delivery system. The drug delivery system may include a container and a dispenser, where the container comprises a luer compartment and a fluid compartment, and where the luer fitting is arranged within the luer compartment and is in fluidic communication with the fluid compartment. In some variations, the dispenser may be directly coupled to the container, while in other variations the dispenser may be indirectly coupled to the container with an adapter that includes a first interface coupled to the container and a second interface coupled to the dispenser. The drug delivery system may include any suitable kind of dispenser. For example, in some variations the dispenser may include an eyedropper. As another example, the dispenser may include a nasal spray pump.

### BRIEF DESCRIPTION OF THE DRAWINGS

FIGS. 1A-1D depict an upper perspective view, a lower perspective view, a longitudinal cross-sectional view, and an exploded view of an example variation of a drug delivery system configured to receive a liquid product through an aseptic transfer.

FIG. 1E depicts a longitudinal cross-sectional view of a container in the drug delivery system depicted in FIGS. 1A-1D.

FIGS. 1F-1H depict a method of transferring a liquid product to the drug delivery system depicted in FIGS. 1A-1D. FIG. 1I depicts the drug delivery system depicted in FIGS. 1A-1H in use for administering a liquid product contained therein.

FIGS. 2A and 2B depict a perspective view and a longitudinal cross-sectional view, respectively, of an example variation of a drug delivery system with an eyedropper.

FIG. 3 depicts a longitudinal cross-sectional view of a container in an example variation of a drug delivery system.

FIG. 4 depicts a longitudinal cross-sectional view of an example variation of a drug delivery system.

FIGS. 5A-5C depict a perspective view, a longitudinal cross-sectional view, and an exploded view, respectively, of an example variation of a drug delivery system with an adapter and an eyedropper.

FIGS. 6A-6C depict a perspective view, a longitudinal cross-sectional view, and an exploded view, respectively, of an example variation of a drug delivery system with an adapter and an eyedropper.

FIGS. 7A and 7B depict a side view and a partially exploded view, respectively, of an example variation of a drug delivery system with a nasal spray pump dispenser.

FIGS. 8A and 8B depict a perspective view and an exploded view, respectively, of an example variation of a drug delivery system with a collar including a luer fitting.

FIGS. 8C and 8D depict a method of transferring a liquid product to the drug delivery system depicted in FIGS. 8A and 8B.

FIG. 9 depicts an example variation of a drug delivery system with a collar including a luer fitting and a luer cover.

FIGS. 10A-10F depict example variations of a drug delivery system having a container configured as a tear-open, single-use eyedropper.

### DETAILED DESCRIPTION

Non-limiting examples of various aspects and variations of the invention are described herein and illustrated in the accompanying drawings.

Described herein are systems and methods for aseptic transfer of a liquid product (e.g., a drug) to a device configured to administer the liquid product in a particular manner. For example, a liquid drug (or other suitable fluid product) may be prepared in a suitable preparation vial (e.g., configured to permit reconstitution of a lyophilized drug, or thawing of a frozen drug such as biologics or small molecules), then transferred to a drug delivery system configured with a product dispenser. The drug delivery system is simple to operate and includes one or more features to help reduce risk of contamination of the liquid product during transfer, as described herein. Additionally or alternatively, the drug delivery system may reduce manufacturing constraints. For example, if a drug were to be stored and transported within its associated drug dispenser beginning from time of manufacture, a customized process may be required to properly fill the drug dispenser with the drug prior to shipping. It would also be challenging to properly sterilize the entire assembled product with the drug and drug dispenser, since the drug and the drug dispenser have different material characteristics and requirements for sterilization (e.g., a typical sterilization process for a drug dispenser may adversely affect the drug). By facilitating an easy way for users (e.g., clinicians or patients at home) to aseptically transfer a drug from a vial to a drug delivery system, the vial and the drug delivery system may be separately assembled and/or sterilized. In other words, the drug delivery system may be sterilized through conventional processes without the drug, while allowing a vial to be filled with the drug and separately sterilized through conventional and well-vetted processes.

Generally, in some variations, a drug delivery system may include a container including a fluid compartment, a luer compartment, a luer fitting arranged within the luer compartment and in fluidic communication with the fluid compartment, and a dispenser in fluidic communication with the fluid compartment. The luer fitting may include, for example, a luer lock with a one-way or resealable valve for aseptically communicating a fluid into the fluid compartment from a source outside the container (e.g., syringe). In some variations, both the fluid compartment and the luer compartment may be internal to the container. However, the luer compartment may be located in any suitable portion of the drug delivery system that is in fluidic communication with the fluid compartment, such as in a separate component that couples to the container. For example, the drug delivery system may include a collar that interfaces between the container and the dispenser, where at least a portion of the luer compartment may be in the collar.

Furthermore, in some variations, the dispenser may be directly connected to the container, or may be indirectly coupled to the container and in fluidic communication via an adapter (e.g., an adapter that converts between different interface types among the container and the dispenser, such as snap fit or threaded interfaces). The dispenser may be any suitable type of drug delivery dispenser, such as an eyedropper, a nasal spray pump, and/or the like.

The drug delivery system may receive a liquid product (e.g., a reconstituted lyophilized product, a thawed product, etc.) via the luer fitting. For example, a syringe containing the liquid product (e.g., drawn from a separate preparation vial) may engage the luer fitting. While the syringe is engaged with the luer fitting, the syringe may be operated by a user to transfer some or all the liquid product from the syringe, through the luer fitting, and into the fluid compartment. After the liquid product is in in the fluid compartment, a user may actuate the dispenser in the drug delivery system to administer the liquid product as appropriate (e.g., as eyedrops, nasal spray, etc.).

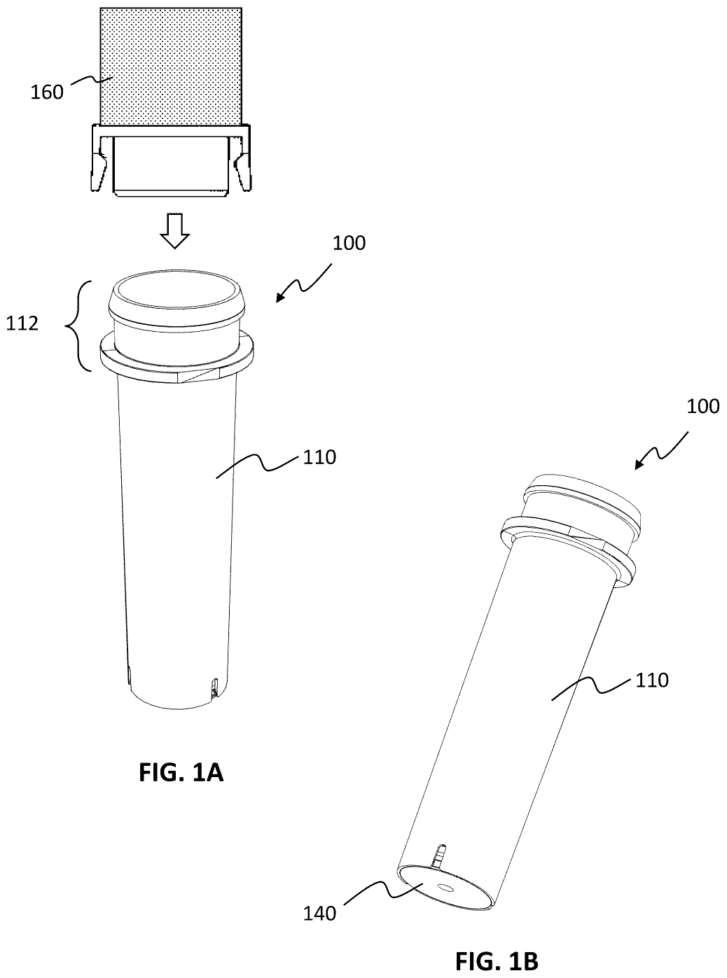

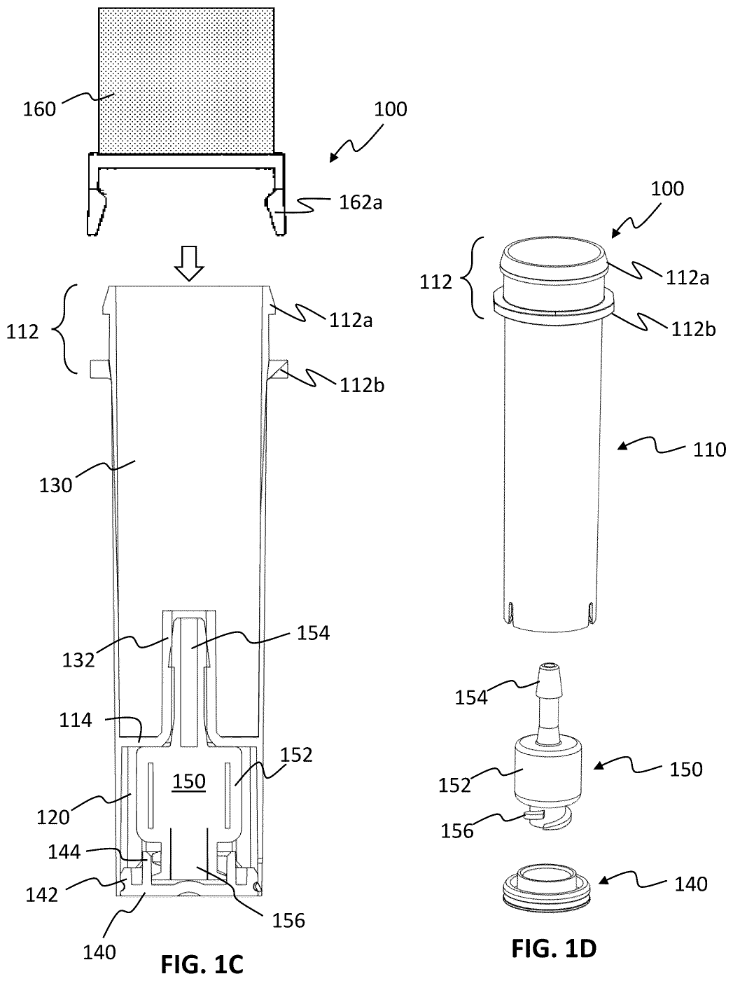

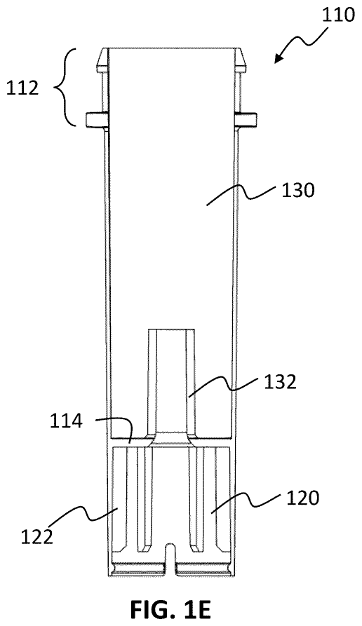

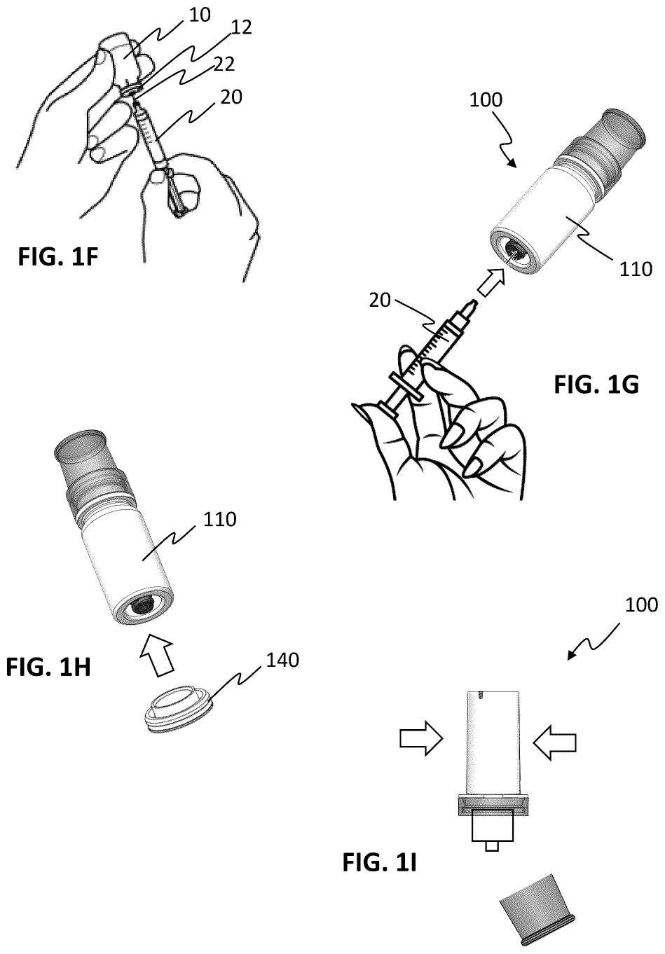

An example variation of a drug delivery system 100 is shown in FIGS. 1A-1D. The drug delivery system 100 may include a container 110 including a luer compartment 120 and a fluid compartment 130 that are internal to the container 110, and a luer fitting 150 arranged within the luer compartment 120 and in fluidic communication with the fluid compartment 130 (as shown in FIG. 1C). The luer fitting 150 may be configured to receive a syringe needle for receiving a fluid. The luer fitting 150 may include, for example, a luer lock with a one-way or resealable valve. As shown in FIG. 1D, the luer fitting 150 may include a hub 152 and one or more connector interfaces configured to engage the fluid compartment 130 and/or other portions of the drug delivery system, such as a luer barb 154 extending from one end of the hub 152, and/or a threaded interface 156 extending from an opposite end of the hub 152. In some variations, the drug delivery system 100 may further include a cap 140 coupled to the container 100 and configured to enclose the luer fitting 150 within the luer compartment.

The drug delivery system 100 may further include a dispenser 160 that is directly or indirectly coupled to the container 110 so as to be in fluidic communication with the fluid compartment 130. The dispenser 160 may, for example, include an eyedropper, a nasal spray pump head, and/or the like, as further described below.

The container 110 is depicted in the figures as generally cylindrical. However, in other variations, the container 110 may have any suitable shape (e.g., tapered, bulbous, pear-shaped, hourglass-shaped, etc.). As shown in FIG. 1C, a portion of the container 110 may include a luer compartment 120 configured to receive a luer fitting 150, and a fluid compartment 130. The luer compartment 120 and the fluid compartment 130 may, for example, be separated by a wall 114. The wall 114 may extend into a conduit 132 that helps facilitate fluidic communication between the luer compartment 120 and the fluid compartment 130. For example, the conduit 132 may include an internal tube that extends from the luer compartment 120, such that fluid may travel from the luer compartment 120 to the fluid compartment 130. In some variations, the fluid may travel from the luer compartment 120 to the fluid compartment 130 within the luer fitting 150, where at least a portion of the luer fitting (e.g., luer barb 154) may be located within the conduit 132. The conduit 132 may have a circular cross-section, or any suitable shape (e.g., corresponding to the shape of the luer fitting). While the conduit 132 is shown as centrally located within the container 110, it should be understood that in other variations, the conduit 132 may be radially offset from a center line or longitudinal axis of the container 110.

As shown in FIG. 1E, the luer compartment 120 and the fluid compartment 130 are axially aligned, with the luer compartment 120 arranged closer to a base portion of the container 110 (e.g., lower end of the container 110, as depicted in FIG. 1E) and the fluid compartment 130 arranged closer to dispenser portion of the container 110 (e.g., upper end of the container 110, as depicted in FIG. 1E). However, in other variations the luer compartment 120 and the fluid compartment 130 may be arranged in any suitable relative manner. For example, the fluid compartment may be in the shape of an annulus, and the luer compartment 120 may be substantially or entirely located within the annulus of the fluid compartment 130 such that the fluid compartment 130 surrounds the luer compartment. As another example, the luer compartment 120 and the fluid compartment 130 may occupy different lateral portions of the container (e.g., a first semi-circular portion of the container 110 may include the luer compartment, and a second semi-circular portion of the container 110 may include the fluid compartment).

The luer compartment 120 may include one or more luer engagement features 122 that engage with the luer fitting 150 within the luer compartment 120. For example, the luer engagement features 122 may include one or more radially inward projections that may help center and/or otherwise guide the placement of the luer fitting 150 within the luer compartment 120. As shown in FIG. 1E, the inward projections may be longitudinal members arranged around an inner surface of the luer compartment 120, forming a cradle that receives the luer fitting 150. The luer engagement features 122 may additionally or alternatively interact with the luer fitting 150 to help lock or retain the luer fitting 150 within the luer compartment. For example, the luer engagement features 122 may include frictional surfaces (e.g., high friction materials), conformable materials (e.g., silicone), textural features (e.g., bumps, ridges), and/or the like. Additionally or alternatively, the luer engagement features 122 may include one or more interlocking features (e.g., lip, shoulder, notch, threads, etc.) that mechanically couple to the luer fitting 150 to help retain the luer fitting 150 in a particular location and/or orientation. As another example, the luer engagement features 122 may additionally or alternatively accommodate a snap fit engagement between the luer fitting 150 and the luer compartment 120. Additionally or alternatively, the luer fitting 150 may be sealed within the luer compartment 120 through a suitable epoxy, an ultraviolet light-cured sealer, and/or the like that is applied around the perimeter of the luer fitting 150 and/or a surface in the luer compartment 120.

As shown in FIGS. 1B-1D, the drug delivery system 100 may further include a cap 140 that functions to protect the luer fitting 150 when not in use (e.g., before and/or after insertion of a syringe into the luer fitting 150). For example, the cap 140 may be configured to couple to the container in a manner that covers or otherwise encloses the luer fitting 150 within the luer compartment 120. As shown in FIG. 1C, in some variations, the cap 140 may be generally disc-shaped and configured to couple to a bottom surface of the container 110 and/or the luer fitting 150. For example, the cap 140 may include an outer annular rim 142 configured to couple to the container 110, and an inner annular rim 144 configured to couple to the luer fitting 150. In some variations, the cap 140 may couple to the container 110 and/or the luer fitting 150 through a mechanical interfit (e.g., via engagement features that couple in a snap fit manner, threaded engagement features, etc.), fasteners (e.g., clips, threaded fasteners, magnets, etc.) and/or any suitable manner. In some variations, the cap 140 may be formed as a separate component that is entirely detachable from the rest of the drug delivery system 100 (e.g., detachable from container 110) as shown in FIG. 1D. Alternatively, the cap 140 may remain coupled to the rest of the drug delivery system 100 (e.g., coupled to the container 110) and movable between an open configuration that exposes the luer fitting 150 and a closed configuration that covers the luer fitting 150. For example, the cap 140 may be coupled to the container 110 at a hinge joint around which the cap 140 may be manipulated between an open configuration and a closed configuration. The cap 140 may be integrally formed with the container 110 (e.g., living hinge, integrated tether), or may be separately formed from and attached to the rest of the drug delivery system 100 (e.g., via a hinge fastener or interlocking features on the cap 140 and the container 110).

In some variations, the cap 140 may provide a tamper-resistant feature for the drug delivery system 100. For example, the cap 140 may be configured such that once it is placed over the luer fitting 150, it is difficult to be decoupled from the container 110 (or moved into an open configuration) to expose the luer fitting 150. Such a tamper-resistant feature may, in some variations, help reduce the likelihood of contamination of contents of the container 110 following fluid transfer therein, by discouraging a user from intentionally or inadvertently reopening the cap 140 and accessing the luer fitting 150 and contents of the container 110. For example, as shown in FIG. 1C, the cap 140 may include smooth, planar surfaces, and/or the cap 140 may engage with the container 110 such that all cap edges are flush with (or recessed relative to) adjacent container edges, thereby making it challenging to grasp and manipulate the cap 140 once the cap 140 is placed over the luer fitting 150. As another example, the cap 140 may be recessed within the luer compartment 120, thereby making it challenging to access the cap 140 once the cap 140 is placed over the luer fitting. As yet another example, the cap 140 may be coupled to the container 110 with one or more fasteners requiring a specialized tool (e.g., Torx fastener) or one-way security fasteners, etc. However, in other variations, the cap 140 may be easily removably coupled to the container 110.

In some variations, such as variations in which the cap 140 forms a base or bottom surface of the overall drug delivery system 150, the cap 150 may include one or more anti-slip features. For example, an outer surface of the cap 150 forming a bottom surface of the drug delivery system 100 may include textural features (e.g., high friction material such as silicone). As such, the cap 140 may help prevent the drug delivery system 100 from sliding or slipping on a surface on which the system is placed.

The fluid compartment 130 of the container 110 functions to receive and store fluid that is transferred via the luer fitting 150 and/or conduit 132. The fluid compartment 130 may have any suitable volume and/or shape for storing a suitable amount of fluid. For example, the fluid compartment 130 may be configured to hold a single dose of drug for a user, or multiple doses of drug for a user.

The container 110 may further include a dispenser interface 112 for coupling to a dispenser 160. For example, as shown in FIG. 1C, the dispenser 160 may include at least one outer member 162a (e.g., a circular member, or multiple arcuate members around the perimeter of the dispenser 160) configured to couple to the dispenser interface 112. Specifically, the outer member(s) 162a may include an angled internal surface that is configured to mate against an angled annular rim 112a on the container 110 in a snap fit manner. To attach the dispenser 160 to the container 110, the dispenser 160 may be pushed onto the dispenser interface until the outer member(s) 162a abut against the annular shoulder 112b and mate with the annular rim 112a in a snap fit manner. Once mated in this manner, the dispenser 160 may be substantially prevented from being moved in the opposite direction due to the ratchet-like features of the outer member(s) 162a and angled annular rim 112a, thereby locking the dispenser 160 in place on the container 110. Although the dispenser interface 112 depicted in FIGS. 1C-1E is one example of a snap fit interface for coupling to a dispenser 160, it should be understood in other variations, the dispenser interface 112 may include other suitable mechanical interfit features providing a snap fit-type interface. Furthermore, in some variations the dispenser interface 112 may include other suitable engagement features (e.g., threads) for coupling to a dispenser 160. The specific nature of the dispenser interface 112 may depend, for example, on the type of interface on the dispenser 160, which also may vary from the example shown in FIG. 1C.

The specific type of dispenser 160 may depend on the specific application of the drug delivery system 100. For example, dispenser 160 may include an eyedropper, or a nasal spray pump. Other characteristics of the drug delivery system may vary depending on the type of application and/or dispenser 160 that is included. For example, in variations in which the dispenser 160 includes an eyedropper, the container 110 may be deformable, so as to allow a user to squeeze the container 110 laterally or radially to drive its contents in suitable droplets out the eyedropper dispenser. The container 110 may, for example, include a flexible material (e.g., silicone) and/or a thin-walled rigid polymer or other suitable material. As another example, in variations in which the dispenser 160 includes a nasal spray pump, the container 110 may be substantially rigid, so as to provide a robust surface that withstands radial handgrip pressure and/or resists buckling when a user actuates the nasal spray pump attached to the container 110. However, even in some variations in which the dispenser 160 includes a nasal spray pump, the container 110 may be deformable similar to that described above, for example if the nasal spray pump dispenser itself provides a sufficiently rigid structure for allowing a user to actuate the nasal spray pump.

Furthermore, in some variations, different portions of the container 110 may have varying rigidity. For example, walls of the container 110 surrounding the fluid compartment 130 may be flexible, while walls of the container 110 surrounding the luer compartment 120 may be rigid. The container 110 may vary in rigidity due to different dimensions (e.g., thin walls that are more flexible than thicker walls), different materials, and/or other construction features.

Generally, the container 110 may be manufactured using any suitable processes, such as injection molding, casting, 3D printing, or other suitable machining processes. Different portions of the container 110 may be integrally formed or may be separately formed and coupled together via mechanical interfit, fasteners, overmolding, and/or the like.

An example method of aseptically transferring a drug to the drug delivery system 100 is illustrated in FIGS. 1F-1I. As shown in FIG. 1F, a syringe 20 may be used to manually withdraw a volume of a drug from a vial 10. In some variations, prior to such withdrawal, the drug may be prepared in the vial 10 (e.g., reconstitution of a lyophilized drug, thawing of a frozen drug, etc.). In some variations, the drug may be withdrawn with the syringe 20 through a needle connected to the syringe that penetrates the septum 12 of the vial. In some variations, the septum 12 may comprise a luer lock adapter so that the syringe 20 is able to withdraw the drug through the luer lock adapter, without a needle 22. As shown in FIG. 1G, the syringe may be coupled to a drug delivery system 100 via an exposed luer fitting 150. The luer fitting 150 may be exposed as shown, either because a cap (similar to cap 140) is not attached to the drug delivery system 100 (e.g., never attached, or was previously attached but removed by the user) or a cap is in an open configuration to allow access to the luer fitting. If the drug was withdrawn from vial 10 via the needle 22 attached to the syringe, the needle may be removed subsequent to withdrawal so that the syringe becomes compatible with the luer fitting 150 comprised in the drug delivery system 100. Once the syringe 20 is engaged with the luer fitting 150, the syringe 20 may be depressed to transfer the drug from the syringe 20 to the drug delivery system 100 through the luer fitting 150. Once all or a sufficient amount of the drug has been aseptically transferred in this manner, the syringe 20 may be decoupled from the drug delivery system 100, allowing the luer fitting 150 to reseal and seal the contents of the fluid compartment in the drug delivery system 100 from external access. As shown in FIG. 1H, the luer fitting may then be covered by coupling a cap 140 to the container 110, thereby enclosing the luer fitting within the luer compartment. Following the above-described process, the drug is stored in the fluid compartment of the drug delivery system 100 and administered in accordance with the dispenser type. For example, as shown in FIG. 1L, an eyedropper cap may be removed to reveal an eyedropper dispenser head, and the container 110 may be squeezed to administer the drug as eyedrops accordingly. Alternatively, a nasal spray pump cap may be removed to reveal a nasal spray tip or nozzle, and the nasal spray pump may be actuated to administer the drug as nasal spray accordingly.

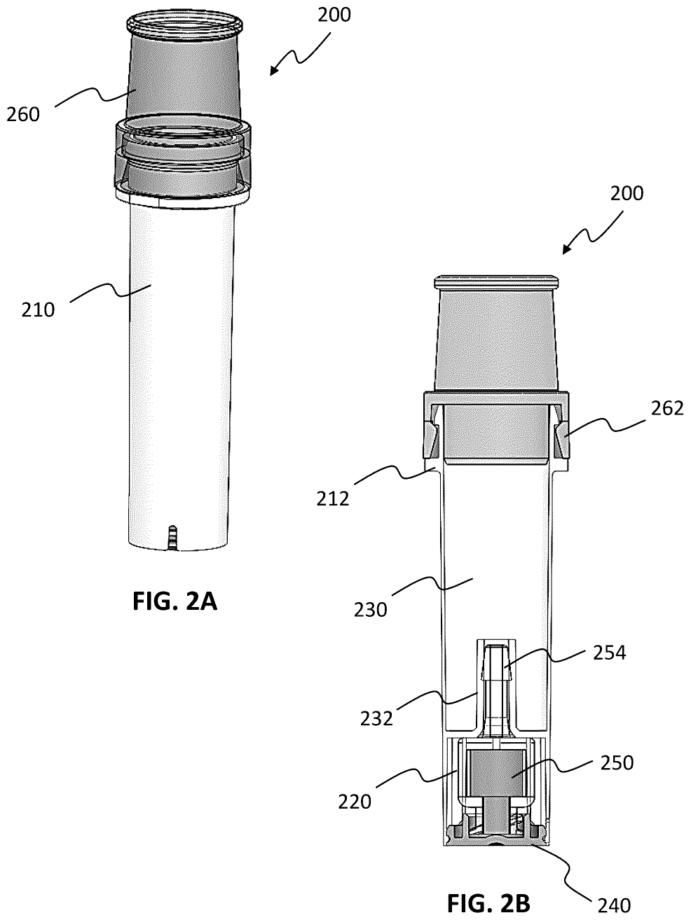

FIGS. 2A and 2B depict an example variation of a drug delivery system 200 similar to drug delivery system 100 described above, configured with an eyedropper. For example, the drug delivery system 200 may include a container 210 that is generally cylindrical and includes a luer compartment 220 located on one end (a bottom end) of the container 210, and a fluid compartment 230 located on the opposite end (an upper end) of the container 210. A luer fitting 250 may be arranged and circumferentially sealed in the luer compartment 220 (e.g., with UV sealant, epoxy, etc.). A luer barb 254 extending from one end of the luer fitting may be arranged in a central conduit 232 that extends from the luer compartment 220 to the fluid compartment 230, and the fluid compartment 230 may surround the conduit 232. The opposite end of the luer fitting may be covered by a cap 240 that couples to the container 210 so as to enclose the luer fitting 250 within the luer compartment 220.

An eyedropper 260 may be coupled to an open end or neck of the container 210 so as to be in fluidic communication with the fluid compartment 230. For example, as shown in FIG. 2B, the eyedropper 260 may couple to a dispenser interface 212 via interlocking features similar to that described above with respect to dispenser 160 and dispenser interface 112 shown in FIG. 1C.

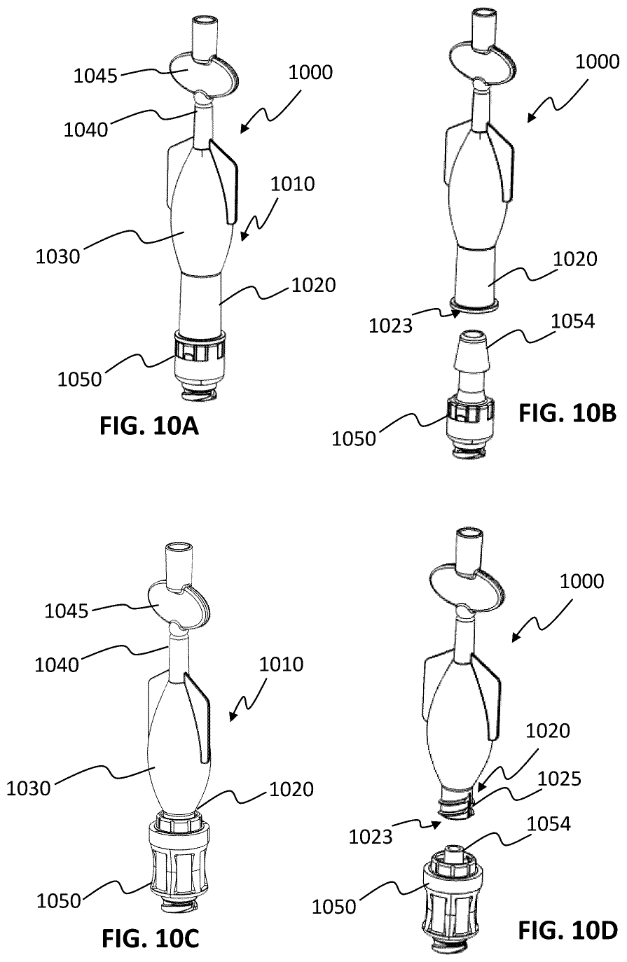

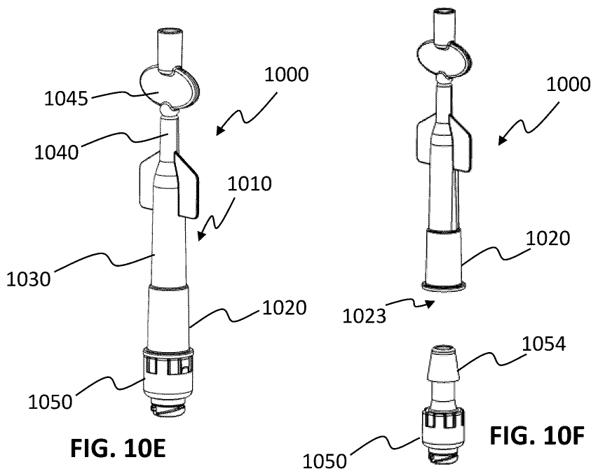

FIGS. 10A and 10B depict an example variation of a drug delivery system 1000 similar to drug delivery systems 100 or 200 described above, except that the container 1010 is shaped and dimensioned as a single-use eyedropper. The container 1010 may include a luer compartment 1020 configured to receive a luer fitting 1050 located on one end (a bottom end) of the container 1010, a fluid compartment 1030 located on the opposite end (an upper end) of the container 1010. The drug delivery system may comprise a dispenser 140 that is connected to or integral with the container 1010 and in fluidic connection with the fluid compartment 1030. The dispenser 1040 may comprise a cap 145 that is tearably connected to the distal end of the dispenser 1040, such that the dispenser and the fluid container 130 is sealed when the cap 1045 is attached, but when the cap is torn off of the dispenser, a liquid content of fluid container 130 may be squeezed out through dispenser 140. A luer fitting 1050 may be arranged in the luer compartment 1020 and a luer barb 1054 extending from the luer fitting 1050 may be arranged in a central conduit 1032 within the luer compartment 1020 to form a fluid connection with the fluid compartment 1030. FIG. 10A depicts the drug delivery system 1000 with the luer fitting attached to the luer compartment 1020, and FIG. 10A depicts the drug delivery system 1000 with the luer fitting detached from the luer compartment 1020, FIGS. 10C and 10D shows alternative version of the drug delivery system 1000 in which the luer compartment 1020 of the container 1010 has an external threading 1025, and the luer fitting 1050 is configured to receive the threading 125, thereby reinforcing the attachment of the luer fitting 1050 to the luer compartment 120. FIGS. 10E and 10F shows alternative version of the drug delivery system 1000 in which the container 1010 shaped and dimensioned as a single-use eyedropper has a thinner fluid compartment 1030 compared to the drug delivery system shown in FIGS. 10A-10D.

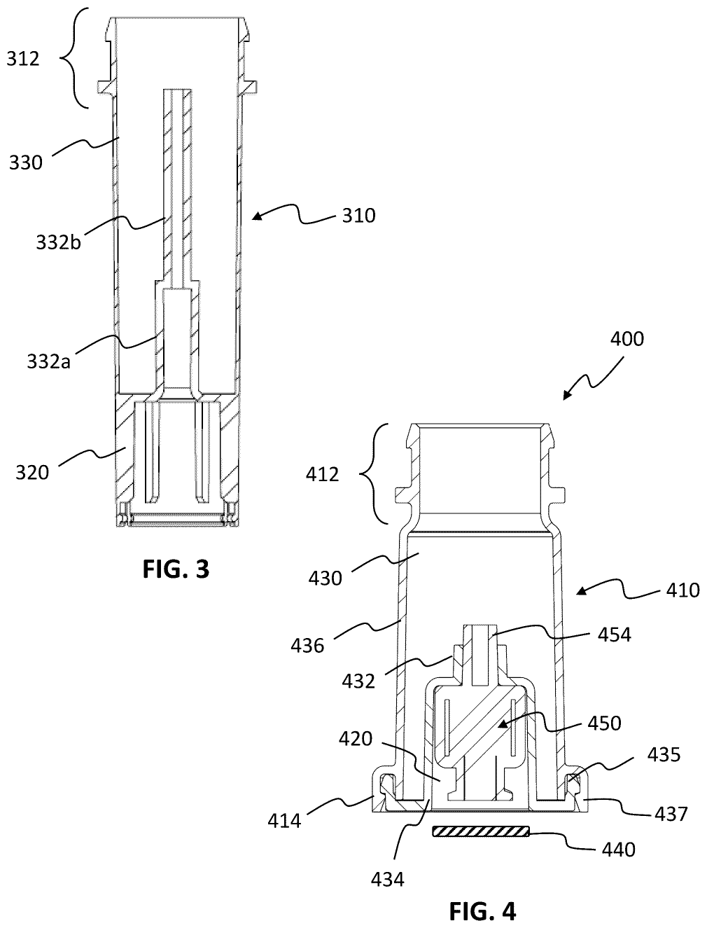

Although specific component dimensions and shapes of the drug delivery system are shown in FIGS. 1A-1I and 2A-2B, it should be understood that in other variations, the overall container, luer compartment, luer fitting, fluid compartment, and/or other related features may have other suitable dimensions and/or shapes. For example, FIG. 3 depicts an example variation of a container 310 in a drug delivery system. Container 310 is largely similar to container 110 shown in FIG. 1E (and includes elements numbered in a similar manner as shown in FIG. 1E), except the container 310 includes an extended conduit having a longer length than the conduit 132 in FIG. 1E. The container 310 may include a conduit having a stepped profile such that the diameter of the conduit varies along its length. Specifically, the conduit may include a first conduit portion 332a that is coupled to the luer compartment, and a second conduit portion 332b extending from the first conduit portion 332a. Compared to the first conduit portion 332, the second conduit portion 332b has a narrower diameter.

As described above, in some variations, a drug delivery system may include a container having a container body formed from multiple container portions coupled together. For example, FIG. 4 depicts an example variation of a drug delivery system 400 including a container 410 that includes a fluid compartment 430 and a luer compartment 420 formed by the joining of multiple container walls. Specifically, container 410 includes an inner container wall 434 and an outer container wall 436. The inner and outer container walls may be coupled at one end of the container, such as at a base of the container (lower end of the container as oriented in FIG. 4). In some variations, the inner and outer container walls may be coupled via mechanical interfit. For example, as shown in FIG. 4, at the base of the container 410, the inner container wall 432 may flare radially outward to form an annular base with an upturned lip 435 or other suitable radial engagement members. The upturned lip 435 may be received within an annular channel 437 extending from the outer container wall 436, and interlocking features within the upturned lip 435 and annular channel 437 may lock the inner container wall 434 and the outer container wall 436 together, such as in a snap fit manner. Additionally or alternatively, other suitable engagement feature(s) coupled to or integrally formed with the inner container wall 434 may couple to corresponding engagement feature(s) coupled to or integrally formed with the outer container wall 436, in order to couple the inner and outer container walls together. Furthermore, in some variations, the inner and outer container walls may be coupled together via one or more suitable fasteners (e.g., epoxy, mechanical fasteners such as screws, magnets, etc.).

Once coupled together, the inner container wall 434 and the outer container wall 436 may collectively define the luer compartment 420 and/or the fluid compartment 430. For example, as shown in FIG. 4, the inner container wall 434 may include a cylindrical surface defining a cavity that functions as the luer compartment 420. The outer container wall 436 may include a cylindrical surface forming the outer surface of at least a portion of the container 410, and the inner container wall 434 may be nested within the outer container wall 436 such the space between the inner and outer container walls functions as the fluid compartment 430. As such, the fluid compartment 420 may have a generally annular shape formed around the luer compartment 420.

A luer fitting 450 may be received and retained in the luer compartment 420, similar to that described above with respect to FIGS. 1A-1E except as described below. In the container 410, a luer end 454 of the luer fitting (similar to a luer barb described above) may be arranged within a conduit 432 but extend beyond the conduit 432 into the fluid compartment 430 itself. The luer fitting 450 may be recessed in the luer compartment 420, leaving clearance for a cap 440 to be placed flush with the annular base of the inner container wall 434. The cap 440 may be coupled to the inner container 434 to cover and enclose the luer fitting 450 within the luer compartment 420.

The open neck portion of the container 410 may include an interface 412 configured for coupling to a dispenser (not shown), such as an eyedropper or nasal spray pump. Similar to that described above, the interface 412 may include one or more engagement features for coupling to the dispenser, such as via mechanical interfit (snap fit, threads, etc.) and/or other attachment methods such as epoxy, etc.

Variations of the drug delivery system described above generally include a dispenser coupled directly to the container. However, in other variations the dispenser may be coupled indirectly to the container by an intervening adapter. For example, in situations where the open neck portion of the container has an interface that is not compatible with the interface of a dispenser, an intervening adapter may enable the indirect coupling of the container and the dispenser such that the dispenser is still in fluid communication with the fluid compartment of the container. For example, the container and dispenser may have different interface types (e.g., one may be snap fit while the other may be threaded, or both may have geometrically incompatible snap fit-type interfaces), or different sizes (e.g., both may be threaded but have different thread pitches, or the container and dispenser may have different diameters). In such variations, the intervening adapter may have one interface that is configured to couple to the container, and another interface that is configured to couple to the dispenser. Accordingly, the adapter may allow a particular container to be operable with a variety of dispenser types (e.g., brands, sizes, etc. of dispensers). Similarly, the adapter may allow a particular dispenser type to be operatable with a variety of containers.

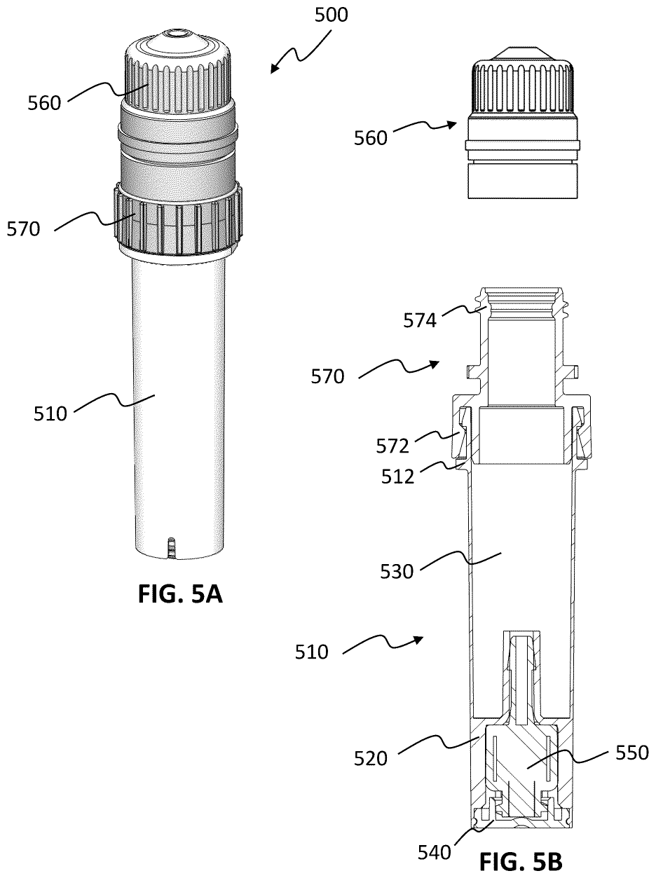

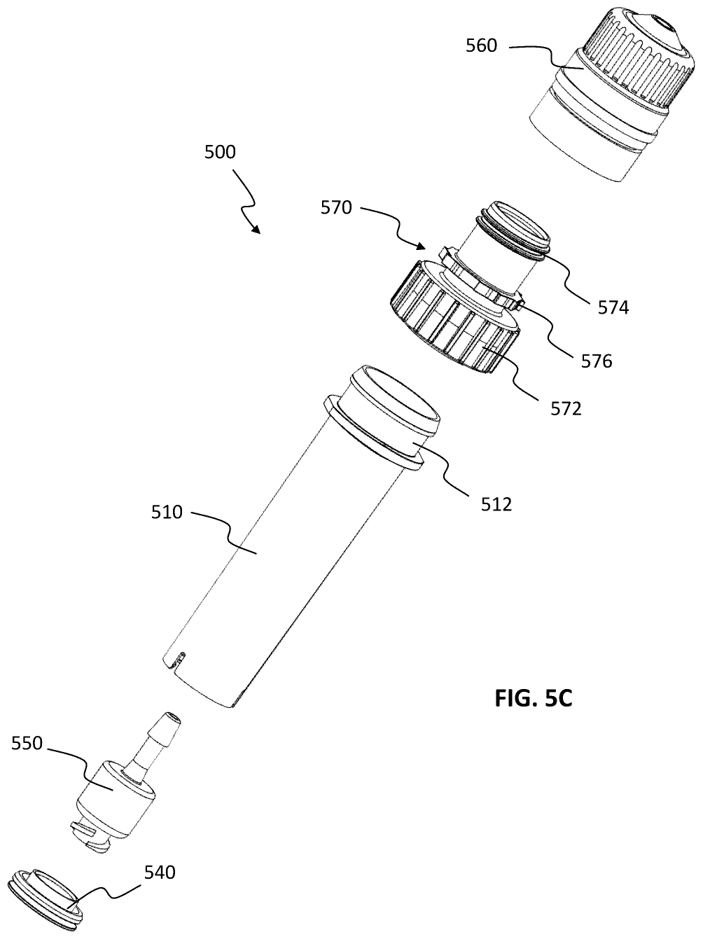

FIGS. 5A-5C depict an example variation of a drug delivery system 500 that includes an adapter converting between a snap fit-type interface and a threaded interface. The container 510 may be substantially similar to the container 110 described above with respect to FIGS. 1A-1E, and with similar numbering. For example, the container 510 may include a fluid compartment 530 and a luer compartment 520 configured to receive a luer fitting 550. A cap 540 may couple to the container 510 to cover and enclose the luer fitting 550 in the luer compartment 520. Furthermore, the container 510 may include a snap fit interface 512 including an annular member similar to interface 112 described above. However, instead of coupling directly to a dispenser, the snap fit interface 512 may be configured to couple to an adapter 570. On its container-side end, the adapter 570 includes at least one outer annular member 572 that engages with the container interface 512 in a snap fit manner. On its opposite dispenser-side end, the adapter includes a threaded interface 574 configured to engage with a corresponding threaded interface of dispenser 560. In some variations, the adapter 570 may further include one or more features that helps lock the threaded engagement between the adapter 570 and the dispenser 560. For example, as shown in FIG. 5C, the adapter 570 may include one or more ratcheted features 576 around its circumference, where the ratcheting features 576 enable one-way rotation of the dispenser 560 relative to the adapter 570 (to couple the dispenser to the adapter) but substantially prevent rotation of the dispenser 560 in the opposite direction (to help prevent removal or decoupling of the dispenser from the adapter).

Accordingly, while the threaded interface of the dispenser 560 is incompatible with and cannot couple to the snap fit interface 512 of the container 510, the dispenser 560 may be attached indirectly to the container 510 via the adapter 570 intervening therebetween. The adapter 570 may include a lumen through fluid in the fluid compartment 510 may be communicated to the dispenser.

In the example shown in FIGS. 5A and 5B, the dispenser 560 includes an eyedropper. However, in other variations the dispenser 560 may include a nasal spray pump or other suitable dispenser. Furthermore, it should be understood that in other variations, the adapter 570 may be configured to interface with any suitable container (e.g., container 310, container 410, etc.) having a snap fit interface that is geometrically compatible with the container-side end of the adapter 570. The adapter 570 may also be configured to interface with any suitable dispenser (e.g., eyedropper, nasal spray pump) having a threaded interface that is compatible with the dispenser-side end of the adapter 570. For example, the specific geometry and dimensions of the adapter's interfaces may vary depending on the specific features of the container and dispenser between which the adapter is intended to be placed.

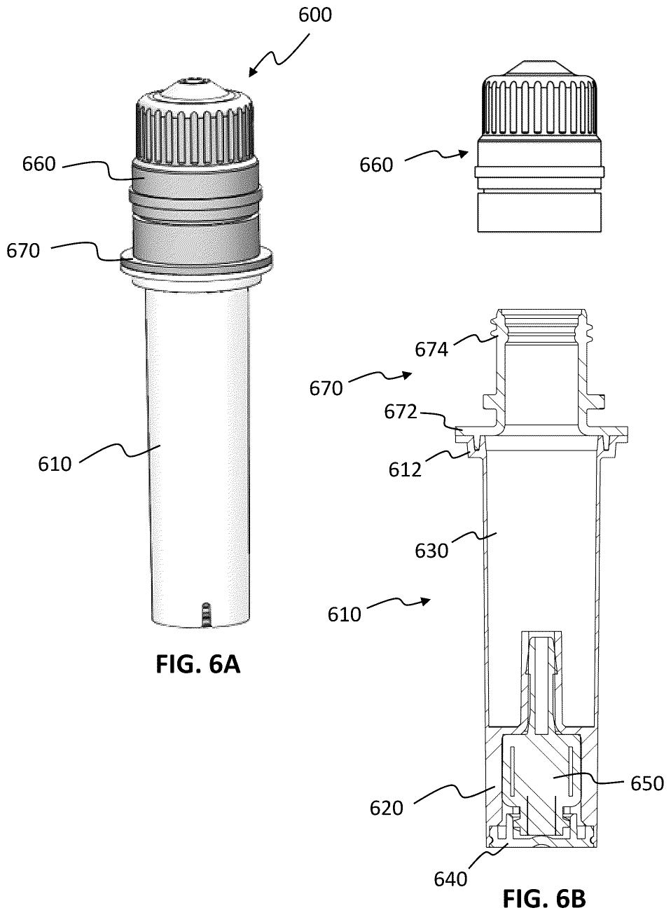

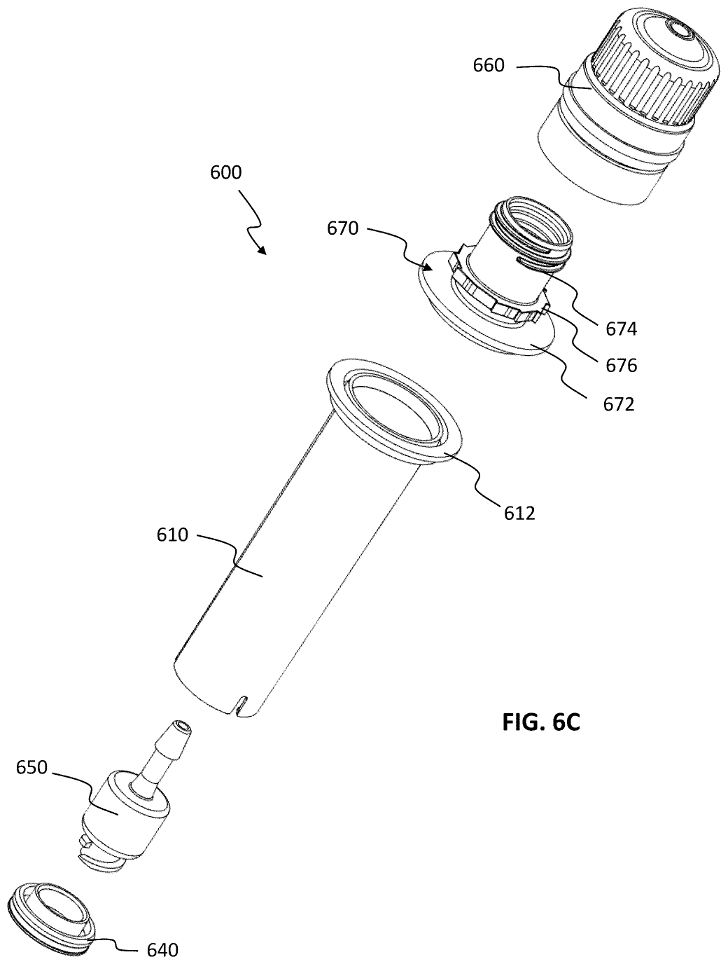

FIGS. 6A-6C depict another example variation of a drug delivery system 600. Like the drug delivery system 500, the drug delivery system 600 may include a container 610 with a fluid compartment 630 and a luer compartment 620 configured to receive a luer fitting 650. A cap 640 may couple to the container 610 to cover and enclose the luer fitting 650 in the luer compartment 620. Similar to the drug delivery system 50, the container 610 itself is not configured to directly couple to a threaded interface of the dispenser 660. Instead, an intervening adapter 670 is configured to engage with the container 610 (e.g., in a direct fitment manner) via mating features that are attached through a suitable mechanical interfit (e.g., snap fit, press fit) and/or with a suitable fastener (e.g., epoxy, mechanical fastener, etc.). For example, as shown in FIG. 6B, the container may include an annular channel 612 that mates with a corresponding annular ring on flange 672 of the adapter 670. On a dispenser-side end of the adapter 670, the adapter 670 may include a threaded interface that couples to the threaded interface of the dispenser 660. In some variations, like the adapter 570, the adapter 670 may further include one or more features that helps lock the threaded engagement between the adapter 670 and the dispenser 660. For example, as shown in FIG. 6C, the adapter 670 may include one or more ratcheted features 676 around its circumference, where the ratcheting features 676 enable one-way rotation of the dispenser 660 relative to the adapter 670 (to couple the dispenser to the adapter) but substantially prevent rotation of the dispenser 660 in the opposite direction (to help prevent removal or decoupling of the dispenser from the adapter).

Accordingly, while the threaded interface of the dispenser 660 is incompatible with and cannot couple to the container 610, the dispenser 660 may be attached indirectly to the container 610 via the adapter 670 intervening therebetween. The adapter 670 may include a lumen through fluid in the fluid compartment 610 may be communicated to the dispenser.

In the example shown in FIGS. 6A and 6B, the dispenser 660 includes an eyedropper. However, in other variations the dispenser 660 may include a nasal spray pump or other suitable dispenser. Furthermore, it should be understood that in other variations, the adapter 670 may be configured to interface with any suitable container (e.g., container 310, container 410, etc.) having an interface that is geometrically compatible with the container-side end of the adapter 670. The adapter 670 may also be configured to interface with any suitable dispenser (e.g., eyedropper, nasal spray pump) having a threaded interface that is compatible with the dispenser-side end of the adapter 670. For example, the specific geometry and dimensions of the adapter's interfaces may vary depending on the specific features of the container and dispenser between which the adapter is intended to be placed.

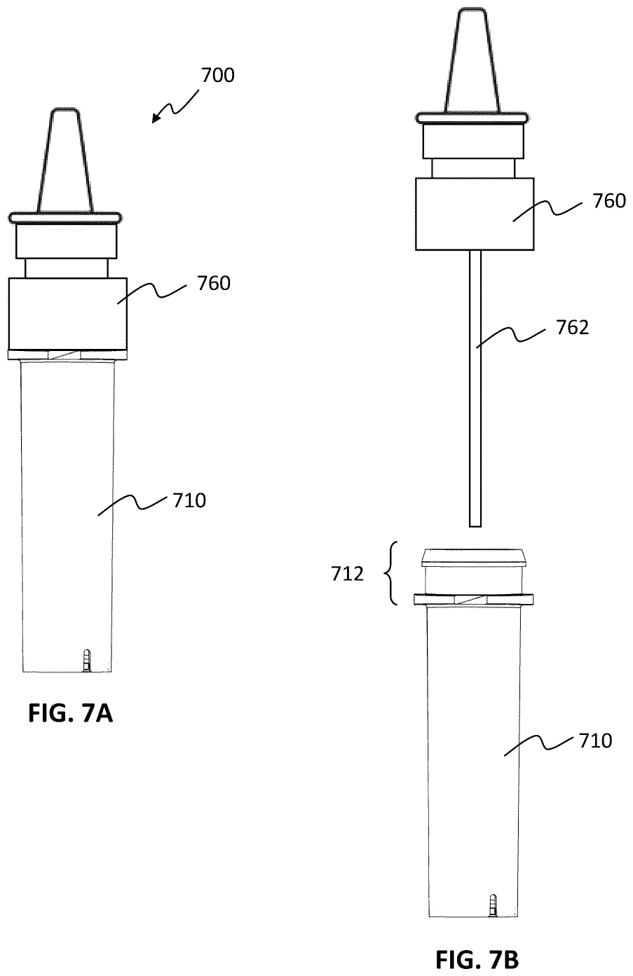

As discussed above, in some variations, a drug delivery system may include a nasal spray pump dispenser coupled to the container. For example, as shown FIGS. 7A and 7B, a drug delivery system 700 may include a container 710 and a nasal spray pump dispenser 760. The container 710 may, for example, be similar to any of the containers described above (e.g., container 110, container 210, container 310, container 410, container 510, container 610, and/or the like). The nasal spray pump dispenser 760 may be configured to couple directly to the interface 712 on the container 710, such as via snap fit, threaded engagement, etc. depending on the type of the interface 712. Alternatively, the nasal spray pump dispenser 760 may be indirectly coupled to the container 710, such as by an adapter (not shown), which may be similar to any of the adapters described above (e.g., adapter 570, adapter 670, etc.). The nasal spray pump dispenser 760 may further include a dip tube 762 of a suitable length and/or diameter for residing in the container 710 once the dispenser 760 is coupled to the container 710.

Although variations of the drug delivery system are primarily described above as including a luer compartment that is internal to the container body, it should be understood that the luer compartment may be located in any suitable portion of the drug delivery system that is in fluidic communication with the fluid compartment, such as in a separate component that couples to the container. For example, the drug delivery system may include a collar that interfaces between the container and the dispenser, where at least a portion of the luer compartment may be in the collar.

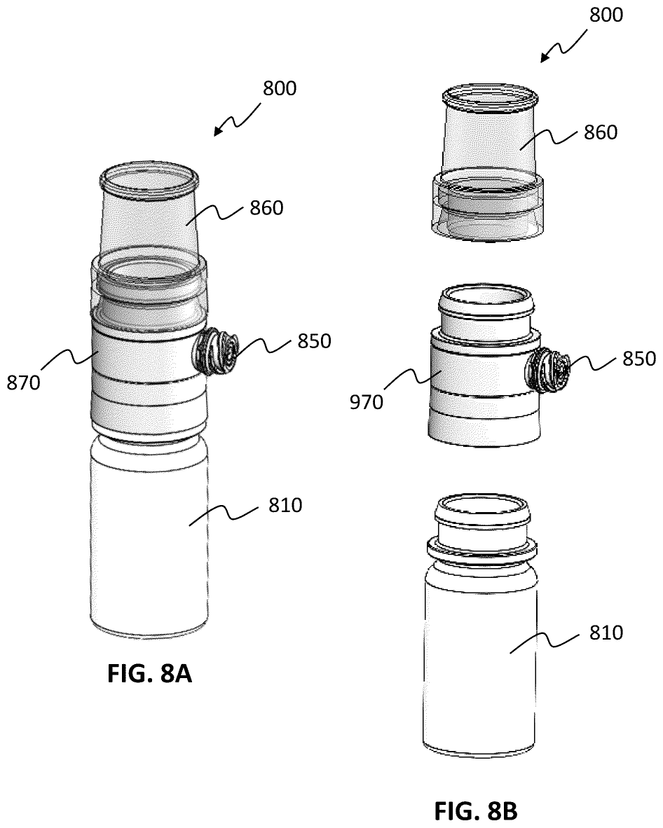

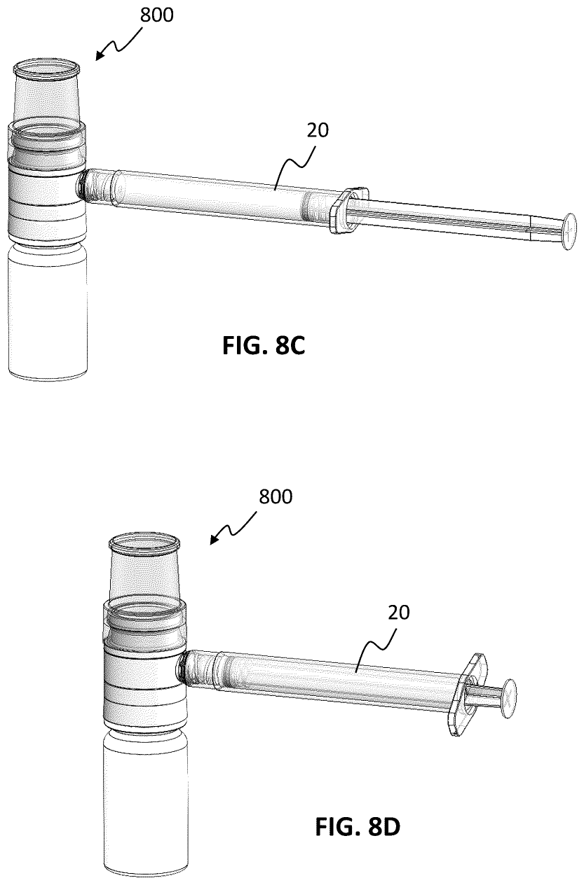

FIGS. 8A and 8B illustrate an example variation of a drug delivery system 800 including a collar 870 including a luer compartment that is configured to at least partially receive a luer fitting 850. A container-side end of the collar 870 may be configured to couple to a container 810, and a dispenser-side end of the collar 870 may be configured to couple to a dispenser 860. The container 810 may, for example, be similar to any of the containers described above (e.g., container 110, container 210, container 310, container 410, container 510, container 610, container 710, and/or the like). The dispenser 860 may include any suitable dispenser such as an eyedropper, nasal spray pump, etc. The luer fitting 850 is shown in FIGS. 8A and 8B partially extending outside of the collar 870 so as to be partially exposed.

As shown in FIGS. 8A and 8B, the luer fitting 950 may be oriented lateral to a longitudinal axis of the container 910 and/or the dispenser 960. Accordingly, the luer fitting 950 may be accessed by a syringe placed laterally to the drug delivery system 900. For example, as shown in FIG. 8C, a syringe 20 may be held lateral to the drug delivery system 800, and a distal end of the syringe 20 may engage the luer fitting (e.g., syringe needle may be inserted into a septum of the luer fitting, distal end of syringe barrel may be threaded onto a threaded end of the luer fitting, etc.). Once the syringe 20 is engaged with the luer fitting (FIG. 8C), the syringe may be actuated to transfer its contents through the luer fitting and into the fluid compartment of the container 810. The syringe may then be detached from the luer fitting. After the fluid has been transferred aseptically to the drug delivery device 800 in such a manner, the exposed end of the luer fitting may, in some variations, be covered by a separate cap (not shown) to protect the luer fitting. The drug delivery device 800 may then be used to administer the fluid contained therein, in the appropriate manner in accordance with the dispenser 860 (e.g., eyedropper, nasal spray pump, etc.).

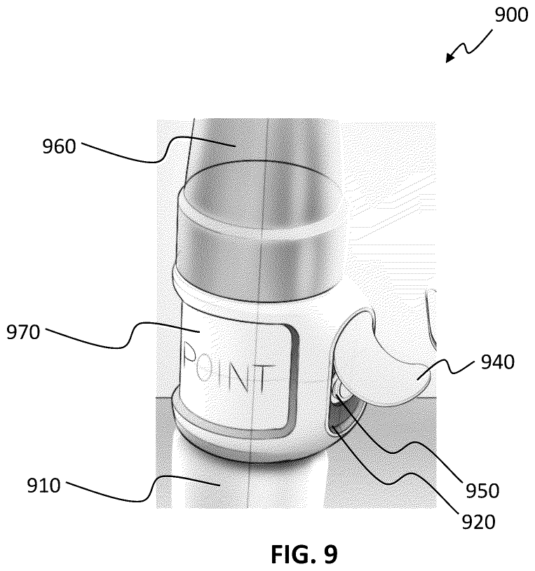

In the figures depicting drug delivery system 800 described above, the luer fitting 850 is partially extending outside of the collar 870 so as to be partially exposed. However, it should be understood that in other variations, the luer fitting may be completely recessed within the collar and/or covered by a cap that may be substantially flush with the collar surface. For example, as shown in FIG. 9, a drug delivery system 900 may include a collar 970 including a luer compartment 920 that is configured to receive a luer fitting 850 therein. A container-side end of the collar 970 may be configured to couple to a container 910, and a dispenser-side end of the collar 970 may be configured to couple to a dispenser 960. The container 910 may, for example, be similar to any of the containers described above (e.g., container 110, container 210, container 310, container 410, container 510, container 610, container 710, container 810, and/or the like). The dispenser 860 may include any suitable dispenser such as an eyedropper, nasal spray pump, etc. Operation of the drug delivery system 900 may be similar to that described above for drug delivery system 800 with reference to FIGS. 8C and 8D.

The luer fitting 950 is shown in FIGS. 9A and 9B entirely within the collar 870, and reversibly covered by a cap 940. The cap 940 may be attached to the collar 970 and movable between an open configuration (exposing the luer fitting 950) and a closed configuration (covering the luer fitting 950). For example, the cap 940 may include a flexible flap. Alternatively, the cap 940 may be a separate component that is removably coupled to the collar 970 to selectively cover a window opening aligned with the luer fitting 950. For example, the cap 940 may include a cover that engages the collar 970 with a mechanical interfit (e.g., snap fit-type interface) or another fastener (e.g., epoxy) to cover the luer fitting 950.

The drug delivery systems 800 and 900 are primarily described above as including a collar that directly interfaces between the container and the dispenser of the drug delivery system. In some variations the collar may further function in a manner similar to the adapters 570 and 670, in that the collar may convert between different interface types on the container and the dispenser that may be incompatible for direct coupling. For example, in situations where the open neck portion of the container has an interface that is not compatible with the interface of a dispenser, the collar may enable the indirect coupling of the container and the dispenser such that the dispenser is still in fluid communication with the fluid compartment of the container. For example, the container and dispenser may have different interface types (e.g., one may be snap fit while the other may be threaded, or both may have geometrically incompatible snap fit-type interfaces), or different sizes (e.g., both may be threaded but have different thread pitches, or the container and dispenser may have different diameters). In such variations, the collar may have one interface that is configured to couple to the container, and another interface that is configured to couple to the dispenser. Accordingly, the collar may allow a particular container to be operable with a variety of dispenser types (e.g., brands, sizes, etc. of dispensers). Similarly, the collar may allow a particular dispenser type to be operatable with a variety of containers.

It should also be understood that yet other variations, the drug delivery system may include additional adapters as appropriate to convert between incompatible interfaces between the container and the collar, and/or incompatible interfaces between the collar and the dispenser.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A drug delivery system comprising:
a container comprising a luer compartment and a fluid compartment;
a luer fitting arranged within the luer compartment and in fluidic communication with the fluid compartment; and
a dispenser in fluidic communication with the fluid compartment.

2. The system of claim 1, further comprising a cap configured to enclose the luer fitting within the luer compartment.

3. The system of claim 1, wherein the luer compartment and the fluid compartment are on opposite ends of the container.

4. The system of claim 1, wherein the container comprises a flexible material.

5. The system of claim 1, wherein the luer fitting comprises a valve.

6. The system of claim 1, wherein the container comprises a conduit between the luer compartment and the fluid compartment.

7. The system of claim 6, wherein the luer fitting is at least partially inserted in the conduit.

8. The system of claim 1, wherein the luer fitting is configured to receive a syringe.

9. The system of claim 1, wherein the luer fitting is coupled to the luer compartment via a fluidic seal.

10. The system of claim 1, wherein the dispenser is coupled to the container.

11. The system of claim 1, further comprising an adapter, wherein the adapter comprises a first interface configured to couple to the container and a second interface configured to couple to the dispenser.

12. The system of claim 11, wherein one of the first and second interfaces comprises a snap fit interface, and the other of the first and second interfaces comprises a threaded interface.

13. The system of claim 1, wherein the dispenser comprises an eyedropper.

14. The system of claim 1, wherein the dispenser comprises a nasal spray pump.

15. The system of claim 1, wherein the container and dispenser are configured as a single-use eyedropper.

16. A method of aseptically transferring a drug to a drug delivery system, comprising:
providing a vial comprising the drug to be transferred;
withdrawing a volume of the drug from the vial with a syringe;
coupling the syringe to a drug delivery system via a luer fitting, wherein the drug delivery system comprises a container and a dispenser, wherein the container comprises a luer compartment and a fluid compartment, and wherein the luer fitting is arranged within the luer compartment and is in fluidic communication with the fluid compartment;
transferring the drug from the syringe to the drug delivery system; and
decoupling the syringe from the drug delivery system.

17. The method of claim 16, further comprising covering the luer fitting with a cap.

18. The method of claim 16, wherein the dispenser is coupled to the container body.

19. The method of claim 16, wherein drug delivery further comprises an adapter comprising a first interface coupled to the container and a second interface coupled to the dispenser.

20. The method of claim 16, wherein the dispenser comprises an eyedropper.

21. The method of claim 16, wherein the dispenser comprises a nasal spray pump.

22. The method of claim 16, wherein the container and dispenser are configured as a single-use eyedropper.
